(19) United States
(12) Patent Application Publication
(10) Pub. No.: US 2009/0325318 A1
(43) Pub. Date: Dec. 31, 2009

(54) SUPRA NANOPARTICLE ASSEMBLIES AND METHODS OF MAKING AND USING THE ASSEMBLIES

(76) Inventor: Keith Carron, Centennial, WY (US)

Correspondence Address: DAVIS, BROWN, KOEHN, SHORS & ROBERTS, P.C.;THE DAVIS BROWN TOWER, 215 10TH STREET SUITE 1300, DES MOINES, IA, 50309, US

(21) Appl. No.: 12/278,118

(22) PCT Filed: Feb. 2, 2007
(86) PCT No.: PCT/US07/02821

§ 371 Date: Feb. 10, 2009

Related U.S. Application Data
(60) Provisional application No. 60/764,489, filed on Feb. 2, 2006.

(51) Int. Cl. G01N 33/553

(52) U.S. Cl. 436/526; 977/774; 977/920

(57) ABSTRACT

A method of reporting a binding event having increased sensitivity and multiplex capabilities. A reporter supra-nanoparticle assembly is provided that has an inner core made of a polymeric material, a coating on the inner core made of a polymeric material and a plurality of reporter nanoparticles, and a first active group on the surface of the coating.

### BACKGROUND OF THE INVENTION

The present invention relates generally to nanoparticles and, more specifically, to supra-nanoparticle assemblies useful as reporters of binding events.

The production of weapons grade nuclear materials requires technical skills and economic resources not usually found in the third world. In contrast, biological agents can be produced inexpensively and without complex facilities. Biological agents can range from human pathogens to agents that destroy livestock or crops. Terror groups that will use biological weapons are not deterred by the threat of a counteroffensive. The best solution to the threat of biological terrorism is a rapid detection system to prevent the spread of agents and to promptly treat those who are infected.

The general purpose of this invention is to provide a reporter for a binding event wherein the reporter provides a very intense signal for trace detection and spectrally distinct signals for accurate identification and multiplexed analysis. Such reporters could be spectrally active Raman dyes or quantum dots (or a combination thereof). An example is the binding event between a stationary antibody in a Lateral Flow Immunoassay (LFI) or a stationary antibody bound to a substrate like a test tube or well in a microtitre plate, or an antibody on a polystyrene particle or paramagnetic particle (such as those described in U.S. patent application Ser. No. 11/211,325 entitled “Paramagnetic Particles For Ultra-Trace Detection” and incorporated herein by this reference), and the antigen and an antibody on a reporter particle (called “Supra-Nanoparticle Assembly—SNA”, as described herein). The binding would indicate the presence of the antigen and could be analyzed by a spectroscopic system. Such analysis could be improved by having the spectroscopic system sampling a spatially localized conglomeration of reporter molecules; such reporters could be localized on test strip or control strip in a lateral flow immunoassay or by a magnetic field in an aqueous solution (when bound to a paramagnetic particle).

Another example of a binding event is the binding between complementary nucleic acid pairs on an SNA to produce a recordable event. Yet another example is the binding of a chemical species to a coating on the SNA.

There are two preferred embodiments of reporter particles within the scope of this invention. One embodiment is a nanoparticle consisting of a polymer core, coated by quantum dots (the reporter) and further coated by more polymer to make the SNA. Receptors specific to the analyte of interest can then be bound to this nanoparticle (primarily through absorption). A second embodiment is a nanoparticle that contains a spectrally active Raman dye associated with a signal-enhancing metal surface and coated with receptor. Both types of these reporter nanoparticles are referred to as “Supra-Nanoparticle Assemblies” (SNA's).

There are currently a limited number of handheld emission spectrometer systems for analyte assays on the market. Smiths Detection of Danbury, Conn. (in partnership with DeltaNu LLC of Laramie, Wyo.) manufactures the leading Raman system. This system identifies chemicals in less than one-minute. It very accurately identifies chemicals or mixtures of chemicals. However, the complexity of biological materials such as bacteria or viruses produces Raman signatures that are indistinguishable. In addition, highly toxic compounds like botulinum toxin or ricin are lethal in quantities that are undetectable with conventional Raman spectroscopy. The problems that need to be solved include enhanced selectivity to distinguish between microbes and enhanced signal to detect trace amounts of materials.

Surface Enhanced Raman Scattering (SERS) from noble metal nanoparticle surfaces amplifies Raman signals from adsorbed molecules by as much as 10¹⁴. This scale of enhancement is sufficient to detect trace amounts of materials, but is not practical because, prior to the instant invention, there was no way to physically proximate the biological target and the SERS surface in a fashion such that the pair can exploit the SERS effect. It also does not solve the problem of similar signatures from biomaterials.

We are proposing to solve both of these problems with a novel assay format that uses SNA's and will have the high specificity of receptor-analyte interactions and the sensitivity of different reporters available today (SERS-enhanced Raman dyes, quantum dots) with the added benefit of simultaneous multi-analyte analysis.

SNA's of preferred embodiments of the present invention can be used in any conventional assay format. For example, a more sensitive Lateral Flow Immunoassay (LFI) can be developed; an aqueous-based immunoassay with capture antibodies absorbed to a substrate can be made; etc. Moreover, the form factor of these formats can be altered to create a long-term continuously monitoring system.

### SUMMARY OF THE INVENTION

A preferred embodiment of the present invention consists of a synthetic procedure to create supra-nanoparticle assemblies (SNA's) that can be used to report a binding event with a target of interest. The supra-nanoparticle assemblies are made with a core of polymer (for example, polystyrene) and a layer of polymer and nanoparticles. Many variations can be made on the central theme. One variation is to use sulfhydryl groups on a polystyrene core to anchor the Au nanoparticles prior to the further polymerization of more polystyrene around the whole construct. The polymer shell could be modified with reactive groups to anchor antibodies are to bind with analytes. Another modification is the addition of a Surface Enhanced Raman Scattering (SERS) enhancer, for example by the addition of halides. It is believed that the halides cause nanoparticle aggregation and molecules bound to the surface become subjected to enormous electric fields between the aggregates. This invention may be particularly useful for this scenario since in practice the aggregation is very hard to control and rapidly leads to species that do not exhibit strong enhancements. In other words, the enhancement by chloride is a fleeting effect and unless stabilized by placing the aggregates in a polymer, it rapidly is lost.

The advantages and improvement over existing methods include a dramatically improved sensitivity because of the spectral intensity of numerous quantum dots or the SERS effect amplifying Raman signals (when using Raman dyes) and unique spectra offered by various reporters. Both of these improvements leads to the possibility of multiplexing assays with multiple-distinct assemblies of reporters. Multiplexing is the holy grail of immunoassays as it permits several analytes to be detected at one time.

An alternative embodiment of the present invention will allow a rapid handheld method to perform assays for biological targets. This goal requires a system that is small, selective, sensitive, and one that ultimately makes multiplexed and continual fast measurements. The technique is termed Paramagnetic Capture Immunoassay (PCI).

### BRIEF DESCRIPTION OF THE FIGURES

FIG. 1 is a drawing of a typical LFI test strip layout.

FIG. 2 is an illustration of Raman nanobarcode concepts.

FIG. 3 is a Raman spectrum of A). SERS active paramagnetic particle, B) Supernatant after application of magnetic field, C) Pellet of SERS active paramagnetic particle and pyridine.

FIG. 4 is a schematic of a handheld PCI biological detection system.

FIG. 5 is an illustration of a procedure for fabricating supra-nanoreporters.

FIG. 6 is an illustration of the design for the handheld biological detection system sample cell.

FIG. 7 is a chart of the anticipated results from a positive PCI assay.

FIG. 8 is a schematic for steps to produce a continuous sampling PCI.

FIG. 9 is illustrates a positive PCI assay.

FIG. 10 is the key for symbols used in FIGS. 13-16.

FIG. 11 is a schematic for a paramagnetic size separation device using the SNAs described herein.

FIG. 12 is a schematic for a paramagnetic size separation device using a laminar flow input.

FIG. 13 is a schematic of the readout portion of the laminar flow paramagnetic size separation device.

FIG. 14 is a schematic for a continuous flow paramagnetic separation device.

### DETAILED DESCRIPTION OF PREFERRED EMBODIMENTS

The particles can be prepared by modifications of the procedures outlined by Wang and Shah in their patent on magnetically responsive polymer particles (U.S. Pat. No. 5,091,206). The differentiation between this patent and theirs is the architecture and content of our supra-nanoparticle assembly. Commonly the reporter is a species that creates a colored pattern for visual detection or fluorescent species for spectroscopic detection. These suffer from poor sensitivity in the case of visual detection and poor differentiation between reporter species in both cases.

### Lateral Flow Immunoassay

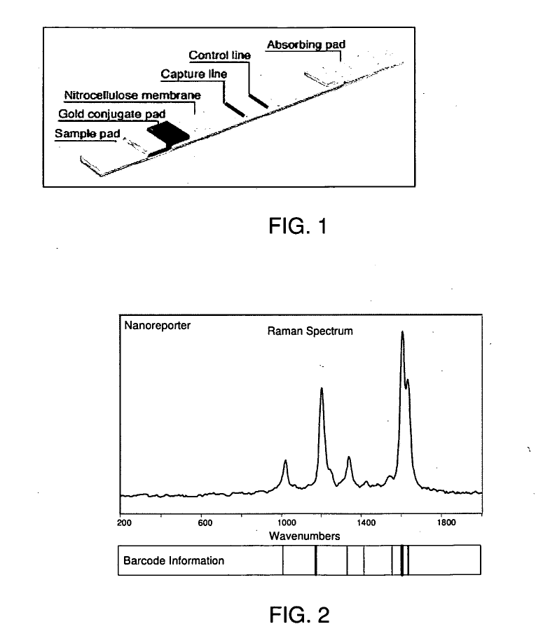

The invention stems from a recent demonstration with Lateral Flow Immunoassay (LFI) which used stationary antibodies and mobile antibodies with a reporter to indicate the presence of an analyte [1]. The format is shown in FIG. 1 generally at 10. A liquid sample is placed at the sample pad 12 on left hand side of the LFI test strip 10 and through capillary action the solution moves past the gold conjugate pad 14. The gold conjugate pad 14 contains mobile antibodies tagged with a reporter. Conventional LFI uses a brightly colored dye or colloidal gold as the reporter. The dyes create a visual pattern that determines the result of the test. The final stage of the assay is a strip or pattern of immobilized antibodies 16. If the mobilized antibodies pick up antigen then the immobilized antibodies will bind to another epitope on the antigen. This leads to a large concentration of reporter at the site of the immobilized antibody and the concentrated reporter can be visualized or detected spectroscopically. An embodiment of the present invention is a new type of reporter that will be more sensitive than existing reporters, which contains more spectral information than fluorescent reporters to produce highly multiplexed assays, and a format that will impart better multiplex capabilities and will be a platform for continual measurement.

### Nanoreporters

A critical missing element in conventional LFI or any other type of immunoassay is the ability to perform simultaneous multiple assays (on a single test strip with LFI or in the same tube or well with aqueous-based immunoassays). This is due to the limited visual acuity to detect several colors and shapes or, in the case of fluorescence, the inability to distinguish between more than 3 or 4 fluorophores. One can conceptually think of the problem in terms of bandwidth of information. The number of frequencies in a fluorescence or absorption curve is very low. Alternatively, the number of frequencies in a vibrational spectrum is very high. A Fourier transform of a spectrum shows the amount of information contained with the different line shape frequencies within a spectrum. A Fourier transform of a typical fluorescence is flat after 1 or 2 frequencies. Raman spectra, on the other hand, contain information over a large range of line shape frequencies. One of the problems to be solved is to create an immunoassay with a reporter scheme that takes advantage of the large information content of vibrational spectroscopy.

An additional problem with LFI is the resistance to flow that builds up during a multiplex experiment. If the multiplexing is performed with several lines of immobilized antibody then, if the first line is positive, a material build-up of reporter occurs and creates a resistance to flow of the second, third, and so on sets of mobilized antibodies. This is a major drawback to the LFI concept.

Several designs for surface enhanced Raman scattering (SERS) nanoparticles have been published [2, 15]. Nanoparticulate gold or silver can be stable for extended periods of time. The stability depends on their Zeta potential [3]. The Zeta potential can be controlled by the monolayer of material coating the particles. Many synthetic schemes use the reduction of metal ions with citrate to produce the particles. In this case the citrate ions adsorb strongly and create a large negative potential. Carron, et al., address stability issues with SERS substrates by using coatings to passivate metal oxidation and to create chemical affinities for analytes at the surface [4]. Coatings containing dyes that exhibit both resonance Raman and SERS produce very intense Raman signals.

Raman spectra vary significantly with the molecular structure of the sample. The same holds true for SERS substrates and monolayer coatings. A useful analogy for understanding the value of a Raman spectrum in various applications is that of a barcode. FIG. 2 represents a novel way of thinking about Raman spectra. A Raman spectrum of a particular analyte is depicted in the figure. Traditionally, Raman spectra have been analyzed for line shapes, line positions, and line intensities. However in the context of this application, Raman spectra can be used simply as reporters for assays. The position and thickness of each line of the Raman barcode corresponds to the position and intensity of the Raman spectrum at a particular frequency. The general theory behind Raman spectral analysis is that each molecule can scatter any combination of wavelengths of light and thus a unique Raman spectrum can be associated with each particular molecule. Therefore, when a Raman spectrum is translated into a barcode, a unique barcode can be correlated to each nanoreporter.

### Raman Instrumentation

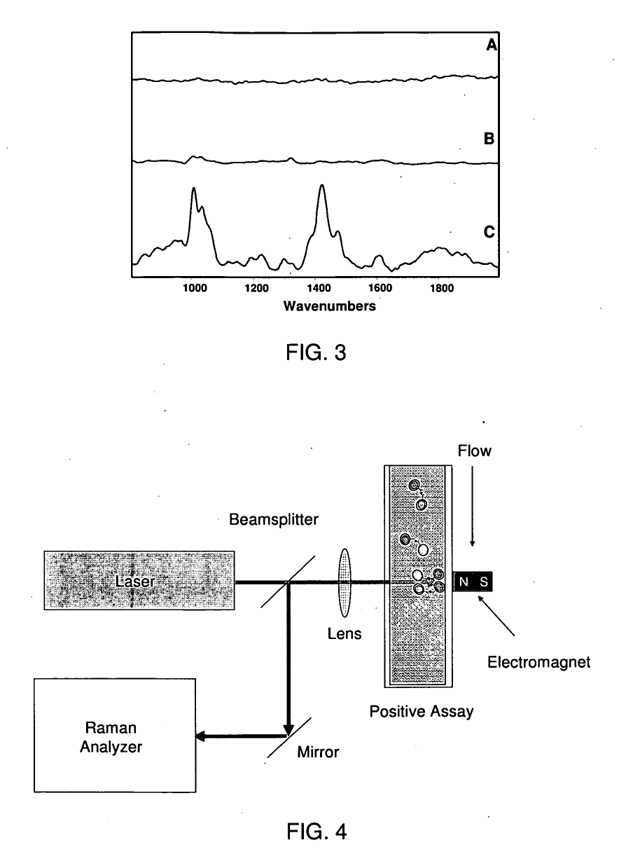

Instrumentation for Raman spectroscopy has undergone dramatic changes in recent years. This has been driven by improvement in CCD quality, laser diodes, and optical filters. These have enabled the production of handheld Raman systems. The first handheld system was the Inspector Raman™ produced by DeltaNu. This system delivers 5 to 40 mW of 785 nm radiation to the sample. It has 10 cm⁻¹ resolution and a range of 200 to 2500 cm⁻¹. The spectrum in FIG. 3 was acquired with the Inspector Raman. This system contains DeltaNu's Watchdog™ calibration feature[5]. This feature maintains calibration such that large libraries of data can be built.

DeltaNu has recently developed a smaller compact system with an embedded computer. This system, supplied to Smiths Detection for marketing, is called the RespondeR RCI™ [6]. The most significant differentiation in the Inspector Raman and, particularly, the RespondeR, is the transition from emphasizing spectral data to emphasizing information. The ultimate result of a scan from the Inspector Raman or the RespondeR is identification and/or quantitation.

### Paramagnetic Particles

Superparamagnetic relaxation is a prominent effect observed in magnetic nanoparticles such as spinel ferric oxide. Interest in applications for these particles has produced many procedures to tailor the size of these particles [7]. This can be performed easily with precipitation or coprecipitation of cations in aqueous solutions. The particles of interest, spinel iron oxides, are easily produced by coprecipitation of Fe³⁺ and Fe²⁺ ions in aqueous solution [8-10]. The size can be controlled from 1.6-10 nanometers by adjusting the pH of the solution during precipitation.

FIG. 3 illustrates a preliminary SERS experiment with paramagnetic particles. The paramagnetic particles were produced using an accepted method of coprecipitation of Fe³⁺ and Fe²⁺ in 6N NaOH at 85° C. FIG. 4 shows the result of modification of these particles with a layer of silver grown on them by the addition of the paramagnetic particles to an Ag⁺ solution and reduction with sodium citrate. Pyridine (0.05 M) was added to act as a reporter on the SERS activity. Raman spectrum A is of the SERS activated paramagnetic particles alone, spectrum B is of the supernatant after a magnetic field has removed all of the activated particles from the solution, and spectrum C is a spectrum of the “plug” of material collected by the magnet field. The large Raman bands in C are associated with adsorbed pyridine. The spectra are all scaled to the same intensity. This demonstration shows that: 1) the activated paramagnetic particles alone do not have a Raman signature; 2) a magnetic field effectively removes the particles from the solution (a small signal is observed from uncoupled SERS particles); 3) the signal is strongly enhanced by the concentration of the SERS nanoreporters by the magnetic field.

Conceptually, one can see how the ultimate limit of detection occurs with free floating nanoparticles. The signal from the surface decreases as the coverage of analyte on the surface decreases. To overcome the decrease in coverage one could decrease the number of silver particles to decrease the active SERS surface. This would make the number of analytes/particle higher. But again a limit is rapidly reached were there are insufficient particles in the laser beam to produce a good signal. The solution to this problem as represented in the present invention is to use a lower concentration of nanoreporters to adsorb a high surface density of analyte and to concentrate them using paramagnetic particles.

### Magnetic Assisted Particle Size Separation

One application of the SNAs is to report a binding event between an SNA of paramagnetic particles coated with receptors to a particular analyte and an SNA of reporter nanoparticles coated with receptors to the same analyte as the paramagnetic SNA. If the analyte is present it will cause the two types of SNAs to bind. The paramagnetic SNA is then used to pull the reporter to a localized spot for spectral analysis.

This approach will yield good sensitivity, but it can be improved on by separating the coupled paramagnetic SNAs from the uncoupled reporter SNAs and paramagnetic SNAs. In order to achieve a quantitative separation, it is necessary to produce coupled species that are significantly different in size from the individual particles. This can be achieved with the large SNA particles described in this invention.

The separation occurs through the effect known as magnetophoresis. A paramagnetic particle is influenced by two forces in the presence of a magnetic field. The first force, F_{mag}, created by formation of an induced magnetic dipole in the paramagnetic particle and the dipole is attracted to the inducing magnet. The second force, F_{drag}, is the drag created by the solution's viscosity. The magnetophoresis, u_{mag}, is described by ratio of these two forces

$${u}_{mag} = \frac{{F}_{mag}}{{F}_{drag}} = \frac{{F}_{mag}}{6 π η {r}_{total}}$$

where η is the solution's viscosity and rₜₒₜₐₗ is the SNA radius. The magnetic force is related to the difference between the susceptibility of the paramagnetic particle and the susceptibility of the surrounding media, the volume of the particle, and the strength of the magnetic field

$${F}_{mag} = \frac{Δ {χ}_{P} \cdot {V}_{P}}{{μ}_{0}} \cdot ( ∇ \cdot B ) \cdot B$$

where χₚ is the magnetic susceptibility of the particle, V_{P} is the particles volume, B is the magnetic field strength, and μ₀ is the permeability of free space. Placing this in the equation for magnetophoresis shows that the product of the magnetic susceptibility and the inverse of the radius of the total species influence the particles motion through a solution in a magnetic field.

u_{mag}∝χ_{P}V_{P}/rₜₒₜₐₗ

This equation shows that a single paramagnetic SNA will travel at twice the velocity of a pair composed of a paramagnetic SNA and a reported SNA. Several scenarios can be developed to use this size dependent magnetophoresis to improve an assay.

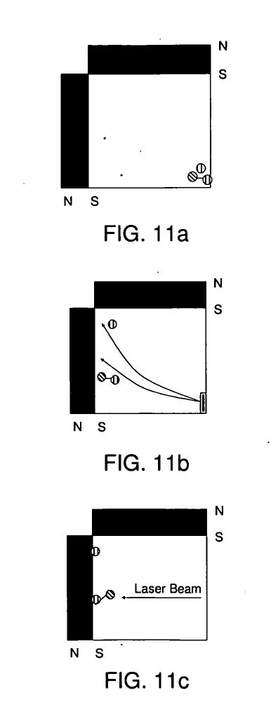

One method for magnetic particle separation uses magnetic fields to create flow and a size dependent deflection. This method uses a reagent container that mixes the reagents (paramagnetic SNAs and reporter SNAs) and allows them to react. Before introduction into a separation device, a strong magnetic field is applied to aggregate the unreacted paramagnetic SNAs and the coupled paramagnetic and reporter SNAs to a portion of the container. Paramagnetic SNAs coupled to reporter SNAs represent a positive and the unreacted paramagnetic SNAs are an interference. The produce a weak background and may occlude reporter SNAs from the laser beam. This initial step is needed to create a packet of particles when the container is placed in the size separation apparatus (FIG. 11a). Two orthogonal magnetic fields are used to move the particles in two dimensions. One dimension is straight across the container and the second, orthogonal dimension, deflects the particles according to their size. After introduction into the separation device the smaller individual paramagnetic SNA particles will be deflected the most and the coupled SNAs will be deflected less (FIG. 11b). Following the separation the particles attached to the side of the container can be analyzed with a laser to read the reporter signal (FIG. 11c).

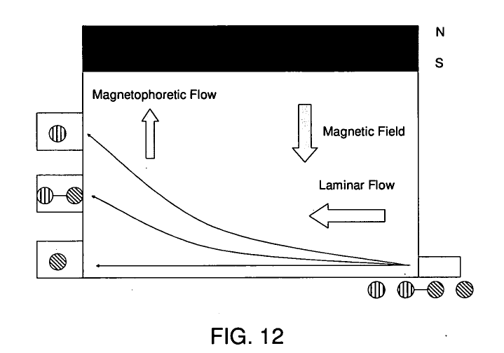

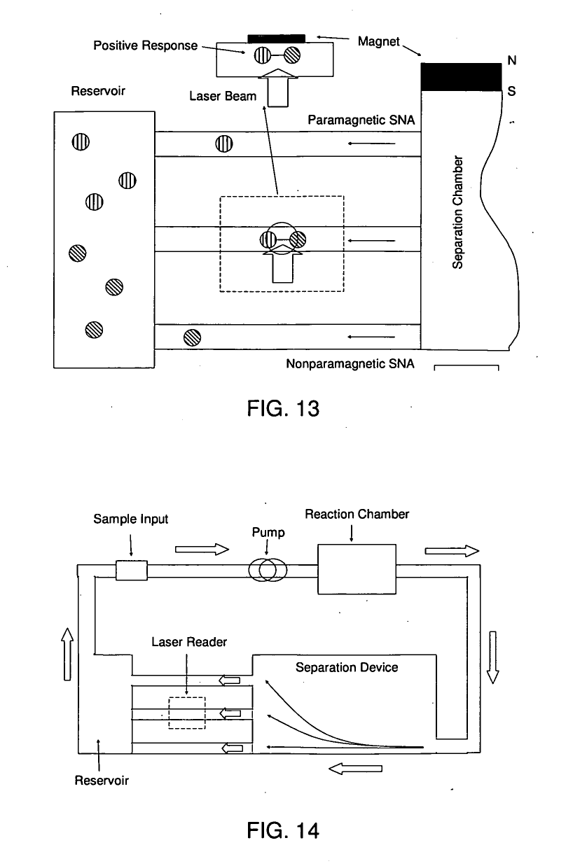

An additional method is comprised of a laminar flow cell (FIG. 12). In this device, a continual flow of material is introduced into the separation chamber. A magnetic field is imposed orthogonal to the laminar flow to create a size dependent magnetophoretic deflection. In this method the separated particles can be collected in flow channels opposite to the input. This method is described by Pamme and Manz for the separation of paramagnetic particles of differing sizes. The significant advantage of this method over the simple separation with two orthogonal magnets is that it also eliminates the non-paramagnetic reporter SNAs which influence the lower limit to detection with an assay as the free floating unbound reporter SNAs will produce a low level background to the assay. In this method, coupled (positive assay) SNAs can be separated from interfering individual paramagnetic SNAs and individual reporter SNAs. The coupled SNAs are gathered by a small magnet in the channel selecting the coupled particles. The ability to collect particles over a period time is a significant advantage over current capillary flow electrophoresis methods that monitor species moving by a fixed observation point. Magnetic collection effectively integrates the assay results to produce a stronger signal than would be achieved by flowing material continuously through the laser beam that induces a signal. The material can be collected in a reservoir after assay for disposal or reuse (FIG. 13).

Another example of the value of a magnetic size separation device is that it an be made into a continuously sampling assay. The laminar flow separation device can be modified with a closed loop pump to remove material from the reservoir back into the separation device. Rather than external agitation, a short loop for mixing and agitation is added to the system. An injection loop is also added to introduce sample (FIG. 14). This test could be used until a positive is recorded or even several prior positives with new positives being recorded as differential signal increases.

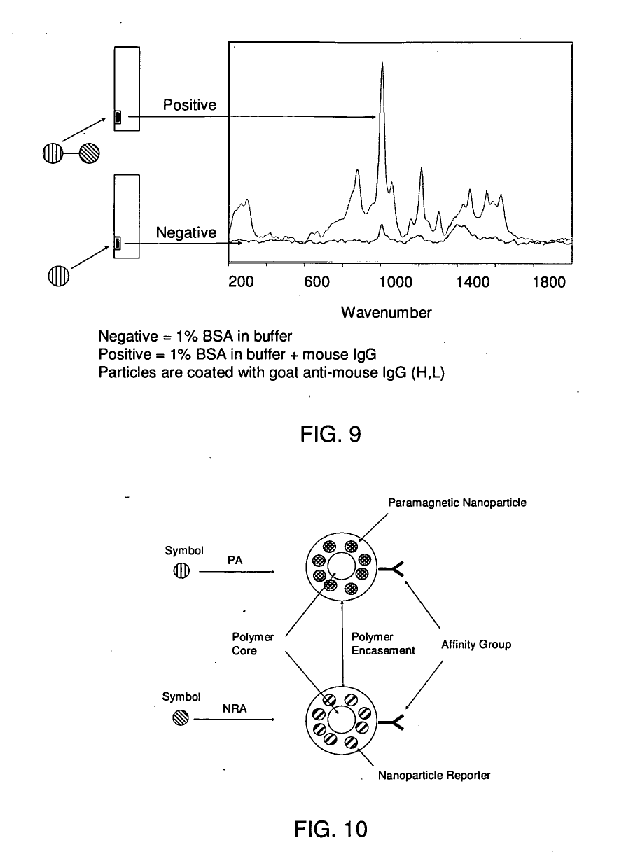

We have demonstrated the coupling of a paramagnetic SNA and a reporter SNA. The SNAs were coated with goat anti-mouse IgG (H,L). It is not ideal to use the same antibody on both particle types as they will compete for the same epitope on the antigen. But this combination sufficed to prove the concept. The reagents were mixed with a control of BSA in a buffer and with BSA, buffer, and mouse IgG (antigen). They were allowed to react for 15 minutes. After the reaction a strong Nd rare earth magnet was used to pull the paramagnetic particles to one side of the vessel. In this experiment the supernatant was removed and Raman spectra were acquired from the pellet of material left behind. We also acquired spectra of the supernatant. The pellet spectra showed a strong signal for the positive test (antigen) present and only a weak signal from the polymer associated with the paramagnetic SNA for the negative (FIG. 9). The supernatant spectra confirmed the test. The supernant from the negative showed a weak signal from the reporter SNAs and no reporter SNA signal from the positive. This confirms that all of the reporter SNAs were taking out of solution by the magnet.

### SUMMARY

LFI is a viable method for handheld detection of biological materials. When Raman SNA's are coupled with LFI it creates a powerful system that uses a small teststrip and a handheld reading device. But, there are significant problematic issues with LFI. First, LFI is not an optimal method for multiplexed assays; rapid response is crucial in first responder situations and a one-step test that produces a response to multiple threats is far superior to repeated testing with an assay format that measures a single antigen. Second, it was developed as a visual test and is not optimal for a sensitive spectroscopic measurement. Third, it is difficult to conceive of a continuous measurement with LFI

The present invention improves on the standard LFI method in several ways. First, is to convert the lateral flow into a fluidic system that provides a platform for multiplexed assays and continual sampling. Second, is to move away from the concepts surrounding a visual response. Most spectroscopic methods operate on the principle of a small focused source of light. Most LFI cards have a ½″ “test” strip. If the test strip were shrunk to the size of a focused laser beam you could instantly gain a signal enhancement of over 100-fold. Our improvement is to use a paramagnetic particle to act as a motive force to spatially localize the nanoreporters in a small, 100 micron diameter spot, to produce an analytical response.

These missing elements in a LFI format are rectified under the teachings of the present invention by using the analyte (antigen) as the coupling agent between a paramagnetic particle and a SERS nanoreporter. The technique differs LFI in that an immobilized antibody is not used. The nanoreporter and paramagnetic particle are free to bind in a flowing solution. The immobilization event is controlled by a magnet. Apparatus for conducting the assay is illustrated in FIG. 4. A paramagnetic flow immunoassay analyzer, indicated generally at 20, includes a positive assay test cell 22 through which the paramagnetic particles will flow. An electromagnet 24 attracts the particles to a location on the wall of the cell 22. The localized particles are illuminated by a laser 26 and the Raman spectrum is detected and analyzed by a Raman analyzer 28. This apparatus is described in U.S. patent application Ser. No. 11/211,325 and referred to as Paramagnetic Capture Immunoassay or PCI [11]. PCI differs from LFI in that the binding event that couples the paramagnetic particle and reporter occurs in solution and immobilization takes place in a magnetic field that is pre-aligned with an optical path for detection. This difference enables designs for rapid analysis of multiple analytes and leads to continuous sampling methods for monitoring applications.

This device could take the form of a handheld Raman system with a small kit that accepts a biological sample from emergency response personnel (when the SNA contains Raman dyes). Or, in the case of SNA's with quantum dots, it could take the form of an emission spectrometer (fluorometer) with the same form as the Smith's Detection RespondeR RCI. An alternative to the simple kit is a continuous sampling device. In this case the magnet is an electromagnet that can be switched on and off to immobilize and mobilize the particles. This leads to a continuous measurement device with a read, followed by an erase capability. The electromagnet is switched on during a read and if an antigen is present and it has coupled the paramagnetic particle to a SNA, a large signal will be observed due to the immobilization of the particle pair in the magnetic field. After the assay is read the magnet is switched off, releasing the particles. If the assay is negative this will release the paramagnetic particles to once again couple with the SNA's if an antigen is present.

Immunoassays according to preferred embodiments of the present invention consist generally of the following components: (a) paramagnetic particle with antibodies for a specific antigen attached to it; (b) a reporter with antibodies attached to it (either a SERS active reporter or a quantum dot); (c) an assay in which the antigen is the binding agent that coupled the paramagnetic particle and the SNA; (d) a magnetic field to localize the paramagnetic particles into a laser excitation source of a handheld emission spectrometer; and (e) a handheld emission spectrometer system to acquire the spectral information and report on the presence or absence of a signal from the reporter on the SNA.

Paramagnetic particles conjugated with antibodies can purchased from Spherotech, Inc., Lake Forest, Ill. They offer coated magnetic particles with a variety of antibody coatings. In a preferred embodiment, nonpathogenic antigens are used. Alternatively, a BSL-IV facility can be used for developing assays for biological weapons of mass destruction. Spherotech's particles are composed of a polystyrene core coated with a paramagnetic coating-copolystyrene layer, followed by a second layer of polystyrene and an antibody coating on top of that. Spherotech offers its magnetic particles over a range of sizes. The smallest size is approximately 400 nm. The SERS active supra-nanoreporters of the present invention will be approximately the same size.

Gold nanoreporters can be synthesized following generally the procedure of Frens that uses a citrate reduction of HAuCl_{4 [}13]. This procedure demonstrates that the particle size can be controlled by the concentration of citrate. Experiments performed in our laboratory indicate that particles between 90 and 100 nm produce the optimal SERS signal. The nanoreporters will be derivatized with a dye that produces a strong surface enhanced Raman signal at the wavelength of the laser excitation of 785 nm for this project. Examples of potential dyes are Nile Blue and copper phthalocyanine tetrasulfonic acid. These particles will be mixed with polystyrene spheres (e.g., 50-100 nm diameter), styrene monomer, and potassium persulfate. This produces a polystyrene-nanoreporter coated polystyrene sphere. This will then be carboxylated with undecylenic acid and cross-linked with divinyl benzene.

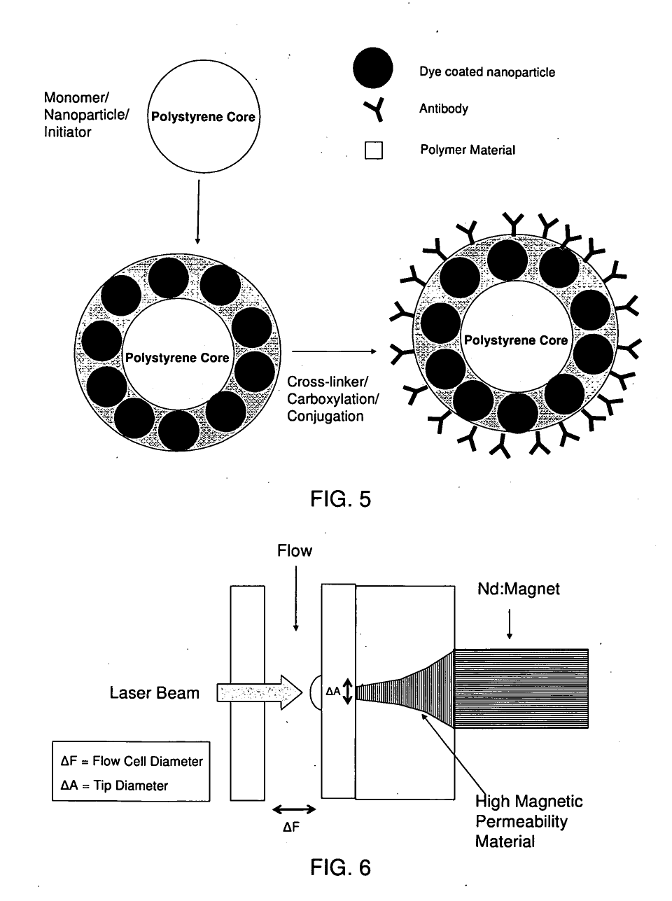

The next step in the production of the supra-nanoreporter structures of the present invention is conjugation with an anti-mouse IgG (FC) antibody using 1-ethyl-3-(3-dimethylaminopropyl) carbodiimide. These procedures follow standard particle coating procedures [12]. The resulting structure is illustrated in FIG. 5. Note, these figures are idealized and do not represent the relative size or topology of the particle elements. The supra-nanoreporters are expected to look more like the SEM of the Spherotech particles.

The Inspector Raman will be modified with a sampling interface for a PCI. The system is fully developed for material identification using Raman spectroscopy. This portion of the project involves fabricating a simple adapter to hold a gravity flow cell and to attract the paramagnetic particles at the focus of the laser beam. A rare earth magnet will be used to generate the magnetic field. The relationship between magnetic field focusing and the sensitivity of the assay will be analyzed. It is expected that the smaller the focus of the magnetic field the higher the sensitivity of the assay due to a higher density of particles in the focus of the laser beam, leading to a larger signal. The magnetic field will be concentrated using a high permeability metal with a taper to focus the field. A range of tapers from 100 μm to 1000 μm will be examined. The lower limit is approximately the size of the focused laser beam at the sample. If the ratio of flow cell diameter relative to the size of the focused magnetic field is large, diffusion of the particles into the magnetic field may be too slow to efficiently capture all of the particles. Both taper and flow cell diameters will be investigated to determine the relationship between the two. Our test apparatus is shown in FIG. 6.

In addition to localizing the particles in a very dense spot, the magnetic field enables optimum alignment of the laser focus with the sample area. A problem with LFI is that it requires the stripe of immobilized antibodies to be printed onto a strip of nitrocellulose. The printing process and the placement of the filter strip in a plastic cartridge lead to a poor tolerance for the location of the strip. This becomes a device problem if the Raman instrument must expand the laser focus, losing sensitivity to account for the intolerance, or must become overly sophisticated to locate the target for each assay. Having the focused magnetic field permanently fixed at the laser focus will eliminate these issues.

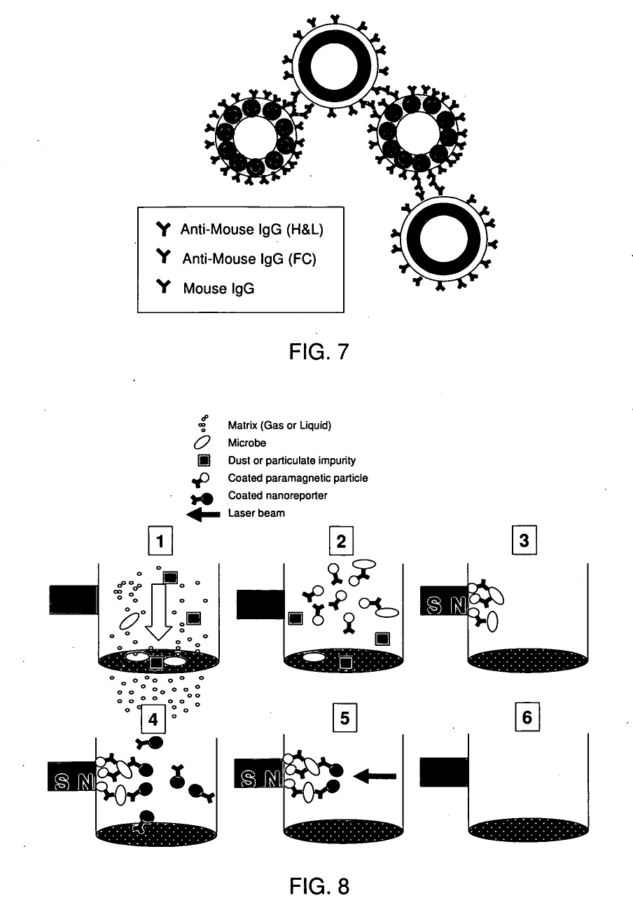

A single analyte assay will be developed using mouse IgG as the antigen. Paramagnetic particles with anti-mouse IgG (H&L) antibodies attached will be purchased and supra-nanoreporters with anti-mouse IgG (FC) antibodies will be fabricated. A diagrammatic presentation of the particle conglomerates formed by this assay are shown in FIG. 7.

Development of this assay will follow standard immunoassay development procedures to find the optimal incubation time and conditions, flow rates through the flow cell, cross reactivity, and sensitivity.

The incubation time and conditions will be tested at room temperature and 37° C. A variety of buffer solutions, including phosphate buffered saline, bicarbonate, and buffers including Tween-20, will be investigated. With uncoated nanoparticles we have found that the buffer composition is very important. Strongly adsorbing anions such as phosphate or chloride strongly affect the aggregation and SERS signal of nanoparticles. We believe that polystyrene coated supra-nanoreporters will be less susceptible to these problems due to their hydrophobicity.

The flow rate is determined by the size of the flow cell or by restrictions within the flow system. As discussed in the previous section, the flow rate will influence the ability of the particle to be localized by the magnetic field. A lower limit will be set by the desired assay time and the upper limit by the strength of the magnetic field. This determination will be important for the design of a prototype with an optimally short assay time for rapid analysis.

Immunological cross reactivity should follow that of the reagent antibodies. A PCI assay is expected to be a viable approach to a handheld biological detection system. Little or no signal is expected to be observed if the binding event shown in FIG. 7 does not occur. If it occurs, it is expected that a large signal from the supra-nanoreporters localized by the magnetic field will be seen. Testing of the anti-mouse IgG system against human IgG will conclusively prove that the signal is generated from an immunological reaction.

Biological threats related to homeland defense may often require trace detection. Sensitivity of the assay is an important parameter in determining the applications for PCI analysis. Standard titer methods will be used to determine the sensitivity for detecting mouse IgG. One can expect a positive result to be an aggregation as illustrated in FIG. 7. This should be similar to detection of a microbe with multiple epitopes since, in the case of trace detection, the reagents will be in large excess and a microbe will represent a surface for aggregation.

The sensitivity is not only related to the immunological event, it is also related to the instrument parameters of laser power, spectral resolution, and integration time. The laser power on the handheld Raman system can be adjusted from 5 mW to 40 mW at the sample. The sensitivity should scale with the laser power, but we have observed laser induced damage to some nanoparticles. Embedding nanoreporters in polystyrene should improve their stability, however, we will examine the laser power vs signal relationship to determine if laser induced damage is occurring.

The Raman system offers three spectral resolution settings. The resolution is set using a Fourier transform apodization of the high frequency (noise) components of the spectrum. As more high frequency components are removed the noise is reduced and the spectral resolution becomes larger. Resolution will be an issue when the PCI concept is expanded to multiplexed assays. The current resolution of our system is 10 cm⁻¹, 15 cm⁻¹, and 20 cm⁻¹. A study by McCreery, et al., indicates that 15 cm⁻¹ resolution in Raman spectroscopy is sufficient to identify more than 1000 compounds in a library search using a correlation search routine [14]. McCreery's study shows that resolution should not be an issue in PCI. We will vary our spectral resolution from 10 cm⁻¹ to 50 cm⁻¹ to establish the relationship between sensitivity and resolution.

The sensitivity in a shot noise limited detection system such as Raman should improve as the square root of the integration time. Integration time can induce laser damage by exposing the supra-nanoreporters to prolonged exposure. Integration time also influences the dynamic range of the detection system. When antigen levels are high a short integration time may be required to prevent saturation of the detector. As antigen levels decrease it will be better to integrate longer to maintain a large S/N. We will develop a software routine that rapidly (<1 second) determines the signal strength and adjust the integration time to maintain a signal of 80% detector saturation. This will extend the dynamic range to include large concentrations and trace detection. The signal reported will be scaled by the integration time.

Two improvements on the single antigen PCI developed in this project will be performed. These are multiplication of the number of antigens that can be detected and using the advantages of paramagnetic capture to develop a system that continuously monitors its environment for microbes, hazardous biological materials, or biomarkers of interest.

The first improvement, a multiplex PCI, can be easily achieved with different adsorbed molecules on our supra-nanoreporters. Previous work by us has shown that 20 to 30 strong resonance Raman active coatings are currently available. Research will be performed to determine their specificity when examined by chemometric methods for spectral differentiation. Multiple reporters types and corresponding paramagnetic particles conjugated with different antibodies will form multiplexed PCIs.

The present invention also includes the following six-step process that will permit PCI to couple with an air or fluid sampling system: (1) Air or fluid is sampled through a watertight, air permeable membrane or a filter to collect microbes; (2) next a solution of paramagnetic particles is added to the filter container and if any microbes with an affinity for the antibodies are present, they will attach to the paramagnetic particles; (3) an electromagnetic is turned on and the paramagnetic particles are collected by the magnetic field and the remaining solution is removed; (4) a solution of supra-nanoreporters is added and if their antibodies have an affinity for the microbes attached to the paramagnetic particles, they will become localized by the magnetic field, and the cell is then rinsed; (5) a laser is used to interrogate what is localized by the magnetic field. If nanoreporters are present a signal will be generated; and (6) the magnetic field is turned off and container is ready to sample again. These steps are illustrated in FIG. 8.

The foregoing description and drawings comprise illustrative embodiments of the present inventions. The foregoing embodiments and the methods described herein may vary based on the ability, experience, and preference of those skilled in the art. Merely listing the steps of the method in a certain order does not constitute any limitation on the order of the steps of the method. The foregoing description and drawings merely explain and illustrate the invention, and the invention is not limited thereto, except insofar as the claims are so limited. Those skilled in the art that have the disclosure before them will be able to make modifications and variations therein without departing from the scope of the invention.

### REFERENCES

1) Presented by Keith Carron at the DARPA Workshop on SERS-Active Nanoparticles, Nanoparticle Assemblies, and Substrates for Chem/Bio Detection; Jul. 29, 2005, Dennis Polla, Organizer.
2) Porous sol-gel silicates containing gold particles as mictures for surface enhanced Raman spectroscopy, Akbarain, et al., J Raman Spectr., 1996, 26:10, 775.
3) Silver colloid particles preparation via reduction by organic redox system, CHEMICA 37, Kvítek Libor, Fichna Petr, Pikal Petr, Novotńy Radko, 1998, 81.
4) Ultrasensitive Detection of Metal Ions with Surface Enhanced Raman Spectroscopy. K. Carron, K. Mullen, H. Angersbach and M. Lanouette, Appl. Spectrosc., 45, 420, (1991).
5) www.deltanu.com
6) www.sensirftp.com/Smiths/Raman/Responder.htm
7) Size Tailoring of Magnetite Particles Formed by Aqueous Precipitation: An Example of Thermodynamic Stability of Nanometric Oxide Particles. Vayssieres, L.; Chaneac, C.; Trinc, E.; Jolivet, J. P Journal of Colloid and Interface Science 1998, 205, 205-212.
8) Influence of iron (II) on the formation of the spinel iron oxide in alkaline medium. Jolivet, J. P.; Belleville, P.; Tronc, E.; Livage, J., Clays Clay Minerals 1992, 40, (5), 531
9) Preparation of aqueous ferrofluids without using surfactant; behavior as a function of pH and counterions. Massart, R., C.R. Acad. Sci. Paris Ser. C1980, 291, (1), 1-3.
10) Elmore, W. C., Ferromagnetic colloid for studying magnetic structures. Physical Review Letters 1938, 54, 309-10
11) U.S. Provisional Appl. No. 60/604,267
12) U.S. Pat. No. 5,091,206
13) Controlled Nucleation for the Regulation of the Particle Size in Monodisperse Gold Suspensions, Frens, G., Nature, 1973, 241, 20.
14) Noninvasive Identification of Materials inside USP Vials with Raman Spectroscopy and a Raman Spectral Library, R. L. McCreery, A. J. Horn, J. S Spencer, E. Johnson, “J. Pharm. Sci., 1998, 87, 1-8.
15) A new approach to determine nanoparticle shape and size distributions of SERS-active gold-silver mixed colloids., Feliidj, et al., J. Sol-Gel Techn., 1998, 725.

We claim:

1. A supra-nanoparticle assembly, comprising:
(a) an inner core comprised of a polymeric material;
(b) a coating on the inner core comprising a polymeric material and a plurality of reporter nanoparticles; and
(c) an active group on the surface of the coating.

2. A method of reporting a binding event, comprising the steps of:
(a) providing a reporter construct comprising a first active group consisting of a reporter supra-nanoparticle assembly as described in claim 1;
(b) providing a capture construct comprising a second active group comprising of a paramagnetic nanoparticle assembly with a second active group on its surface;
(c) introducing the reporter and capture construct into a fluid having target particles with binding sites for the first and second active groups, whereupon both reporter and capture construct bind to the target particles; and
(d) using a detector to detect the concentrated reporter nanoparticle assemblies.

3. A method as defined in claim 2, wherein the second active group is a paramagnetic nanoparticle assembly and the first and second active groups, when bound together, are separated from unbound reactive groups and concentrated by supplying a magnetic field.

4. A method as defined in claim 2, wherein the reporter construct nanoparticles comprise surface enhanced Raman scattering materials.

5. A method as defined in claim 2, wherein the reporter construct nanoparticles comprise quantum dots.
